(19) United States
(12) Patent Application Publication
(10) Pub. No.: US 2004/0243005 A1
(43) Pub. Date: Dec. 2, 2004

(54) Remote speaker microphone having vital sign monitoring capability

(76) Inventor: Gary M. Rapps, Sunrise, FL (US)

Correspondence Address: Barbara R. Doutre, Motorola, Inc., Law Department, 8000 West Sunrise Boulevard, Fort Lauderdale, FL, 33322, US

(21) Appl. No.: 10/447,427

(22) Filed: May 29, 2003

(51) Int. Cl. A61B 5/02; A61B 5/08; A61B 5/103

(52) U.S. Cl. 600/481; 600/534; 600/595

(57) ABSTRACT

A communication system (200) includes a portable radio (202) having as a remote accessory (204) coupled thereto. The remote accessory (204) is preferably a remote speaker-microphone that includes a controller (206) and an accelerometer (208) that monitors a user's vital signs, such as respiration and heart rate. A dispatch station (210) receives the vital sign information from the radio (202) and a detection/notification device (212) detects the vital sign information and notifies support personnel distress conditions.

### TECHNICAL FIELD

This invention relates in general to public safety apparatus, and more particularly to vital sign monitoring through the use of such apparatus.

### BACKGROUND

It has become increasingly important to monitor public safety personnel as they enter into high-risk, dangerous environments. Monitoring these individuals not only protects them physically but also increases the ability of coordinators to strategize how to approach each rescue scene. It would be beneficial to have a non-intrusive device that monitors physical characteristics of public safety personnel, such as heart rate, respiration, and movement, and to have this information transmitted to another place for monitoring. The ability to recognize whether an individual is experiencing distress would facilitate getting the necessary help to that individual in a timely fashion.

All currently available equipment for monitoring heart rate falls into two test equipment categories—Electro-Cardiogram and Plethysmography. Electro-Cardiogram (ECG or EKG) is a test that senses the electrical signal that triggers the heart muscle. Plethysmography provides a measurement of the systolic blood pressure (maximum pressure exerted when the heart contracts) of the lower extremities as compared to the upper extremities. The ECG requires electrodes to be touching a subject's skin, and Plethysmography requires three blood pressure cuffs wrapped snugly around the person's extremities (both biceps and one upper thigh). There are no portable non-intrusive heart monitors currently on the market.

All currently available respiration monitoring equipment requires probes be placed on the person's thorax, neck, torso, nose or mouth. There are no portable non-intrusive respiration monitors currently on the market.

Many firefighters currently wear a Personal Alert Safety System (PASS) device that detect a lack of movement only, but these devices are limited to monitoring one physical characteristic. The PASS device only sends a warning to the individual user, such as through an alarm or lights, but does not notify anybody else. The use of a Man Down switch senses when an individual has switched from vertical to horizontal orientation and sends an alarm signal back to a dispatch station, but no other information is provided.

Accordingly, there is a need for a non-intrusive device and system that provides physical information pertaining to an individual in distress and forwards that information to support personnel.

### BRIEF DESCRIPTION OF THE DRAWINGS

The features of the present invention, which are believed to be novel, are set forth with particularity in the appended claims. The invention, together with further objects and advantages thereof, may best be understood by reference to the following description, taken in conjunction with the accompanying drawings, in the several figures of which like reference numerals identify like elements, and in which:

FIG. 1 shows a block diagram of a remote radio accessory having vital sign monitoring capability in accordance with the invention;

FIG. 2 shows a block diagram of the remote speaker-microphone having vital sign monitoring capability within a communication system in accordance with a preferred embodiment of the invention; and

FIG. 3 shows an example of a graph of measurements of movement and vital sign parameters taken with a remote accessory formed in accordance with the present invention.

### DETAILED DESCRIPTION OF THE PREFERRED EMBODIMENT

While the specification concludes with claims defining the features of the invention that are regarded as novel, it is believed that the invention will be better understood from a consideration of the following description in conjunction with the drawing figures, in which like reference numerals are carried forward.

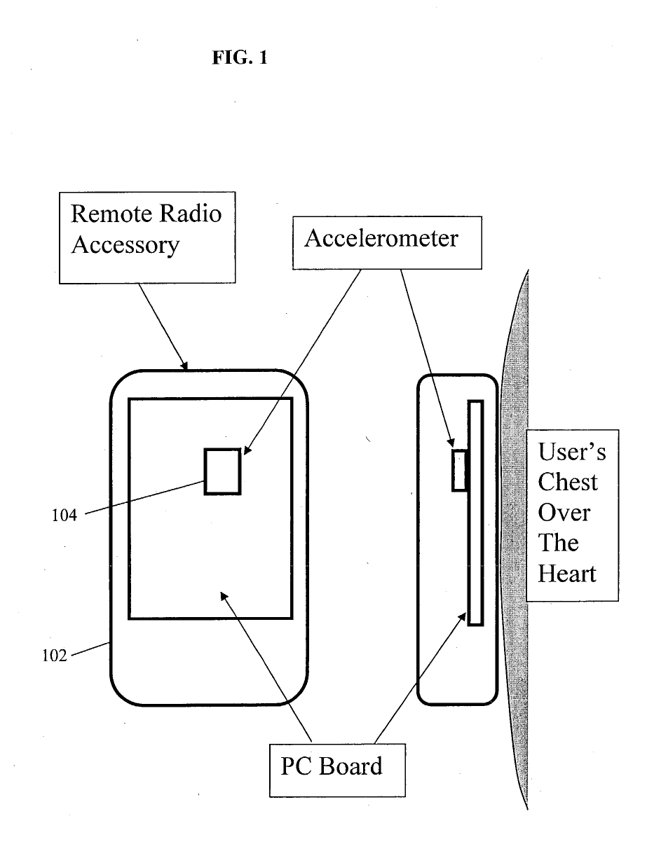

Referring now to FIG. 1, there is shown a front and side view of an apparatus 100 for monitoring vital signs of an individual in accordance with the present invention invention. The apparatus 100 comprises an accessory for a portable radio and in the preferred embodiment, consists of a remote speaker-microphone 102. In accordance with the present invention, coupled to or within radio accessory 102 is an accelerometer 104, the accelerometer for monitoring an individual's vital signs. In accordance with the invention, the accelerometer 104 monitors the individual's movement and at least one of the individual's heart rate and respiration. For the purposes of this application, vital signs include, but are not limited to, heart rate and respiration. In accordance with the present invention, proximate coupling of the radio accessory 102 to the user's body allows the accelerometer 104 to monitor the user's vital signs. The radio accessory 102 is proximately coupled to the user's body by wearing the accessory on the body, such as clipped to the chest or held by a holster worn close enough to the body so that the accelerometer detects movement. Alternatively, radio accessory 102 may be integrated within a garment, such as a coat, that enables sufficient coupling for the accelerometer 104 to monitor an individual's vital signs. As long a movement can be detected by accelerometer 104, then a user's vital signs can be determined as will be described below.

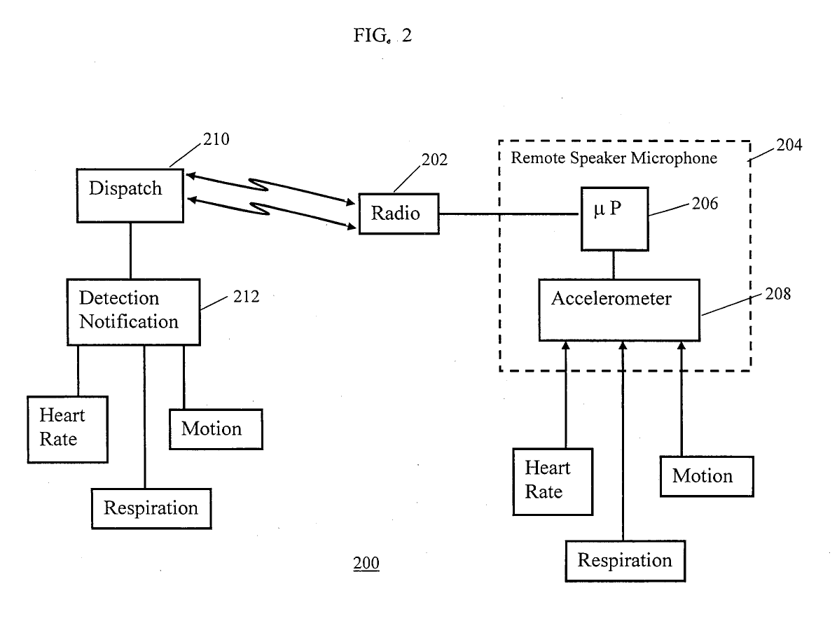

FIG. 2 shows a block diagram of a radio accessory having vital sign monitoring capability operating within a communication system in accordance with a preferred embodiment of the invention. Communication system 200 includes a portable radio 202 worn by a user (not shown), a remote accessory 204 coupled to the portable radio, and a dispatch station 210 having a detection/notification device 212 coupled thereto. The remote accessory 204 includes a controller 206 and an accelerometer 208 coupled to the controller for monitoring the user's vital sign information. The dispatch station 210 receives the vital sign information from the radio 202 and the detection/notification device 212 detects the vital sign information and provides notification of the vital sign information to others, such as support personnel. In accordance with the invention, the accelerometer monitors movement and at least one of heart rate and respiration. Thus, the communication system 200 of the present invention provides valuable vital sign information by utilizing existing accelerometer technology.

In operation, the accelerometer 208 monitors in addition to movement, at least one of a user's vital signs, such as heart rate and/or respiration, and transfers this information to the radio 202 via controller 206. The radio 202 transmits the vital sign information over to the dispatch station 210 where it is detected by detection/notification device 212 and notified to others. A response team can, in turn, react to physical conditions of rescue personnel. The dispatch station 210 can also make inquiries of and/or send information to the individual wearing the accessory. Even if the user is non-responsive, the dispatch station 210 can enable the monitoring features of the remote accessory 204 via controller 206. For example, if an individual is not moving for a predetermined period of time indicating a certain level of distress, the dispatch station 210 can enable the heart rate and respiration monitoring features of the remote accessory 204 so as to provide valuable information on the present condition of that individual. Even if the subject is moving, the additional cardiovascular and respiration monitoring capability can be used to facilitate curtailing a distress situation before it happens. Physical profile characteristics can be created to study the impact of various stressful environments on public safety personnel thereby providing long term benefits to the public safety arena.

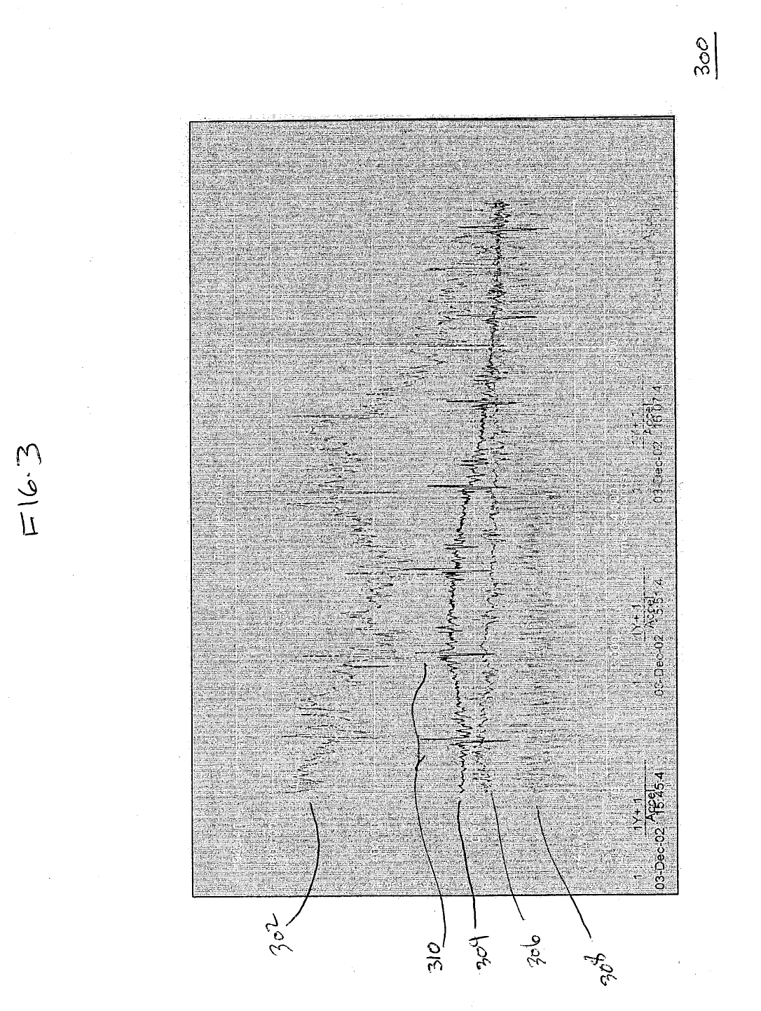

FIG. 3 shows an example of a graph of measurements of movement and heart beats superimposed thereon taken with a remote accessory formed in accordance with the present invention. Graph 300 shows waveforms representing movement under various conditions. Waveform 302 represents slow shoulder movement. Waveform 304 represents lying on the back. Waveform 306 represents kneeling (still). Waveform 308 represents talking while standing still. Movement waveforms 302-308 were measured using an accelerometer located within a remote speaker-microphone mounted on a person's chest.

Waveform 304 shows heart beats 310 at normal intervals within the movement waveform. The heart rate can be determined by filtering out the beats 310 within the movement waveform 304. Respiration data can be obtained in a similar manner. Vital sign monitoring is based on using the accelerometer to measure and quantify human heart rate and/or respiration rate for the primary purpose of distress detection.

The remote speaker-microphone is preferably mounted such that the accelerometer is located in the plane that is most sensitive to the vital sign parameter being measured. Although the pulse of the heart beat can be detected in three axes, the accelerometer does not need all three of these to detect this parameter. Accordingly, the accelerometer is capable of detecting heart beats with the remote speaker-microphone mounted on the user's chest or a variety of other positions.

The accelerometer used in accordance with the present invention serves two purposes. First, the accelerometer quantifies human movement to establish a movement/no movement event threshold. If the magnitude of this event is outside of a predetermined threshold, indicating a no movement event, then a signal is sent to the dispatch station to notify support personnel. Second, the accelerometer is used to determine the present vital sign characteristics of the user, including but not limited to heart rate and respiration rate. Filtering of the accelerometer's movement waveforms leave heartbeat and respiration waveforms for identifying and quantifying these vital sign parameters. The vital sign parameters are then reported back to support personnel. These synergetic operations provide a non-intrusive means of determining the user's well-being.

Accordingly, there has been provided an apparatus and system that monitors an individual's vital sign information in a public safety environment and forwards that information to support personnel. The ability to monitor heart rate and respiration in addition to movement provides additional information about a subject's well being which facilitates a timely response from support personnel when needed. The apparatus and system of the present invention can be used in all public safety disciplines, such as fire fighting, rescue, police, paramedic, and any other job where vital sign monitoring are important for the well being of individuals.

While the preferred embodiments of the invention have been illustrated and described, it will be clear that the invention is not so limited. Numerous modifications, changes, variations, substitutions and equivalents will occur to those skilled in the art without departing from the spirit and scope of the present invention as defined by the appended claims.

What is claimed is:

1. An apparatus for monitoring vital signs, comprising:
a remote speaker-microphone; and
an accelerometer coupled to the remote speaker-microphone, the accelerometer monitoring the vital signs.

2. The apparatus of claim 1, wherein accelerometer monitors at least one of heart rate, respiration, and movement.

3. The communication system of claim 1, wherein the accelerometer monitors movement and the vital sign information is derived therefrom.

4. A remote speaker-microphone, comprising:
a controller; and
an accelerometer coupled to the controller, the accelerometer monitoring an individual's vital signs.

5. The remote speaker-microphone of claim 4, wherein the remote speaker-microphone is proximately coupled to the user's body.

6. The remote-speaker-microphone of claim 5, wherein the remote speaker-microphone is located within a coat.

7. An accessory for a portable radio, comprising:
a controller;
an accelerometer coupled to the controller; and
the accessory coupled to the portable radio, the accessory being proximately coupled to the user's body for monitoring the user's vital signs.

8. The accessory of claim 7, wherein the accelerometer monitors at least one of heart rate, respiration, and movement.

9. The accessory of claim 7, wherein the accelerometer monitors movement and at least one of heart rate and respiration.

10. A communication system, comprising:
a portable radio worn by a user;
a remote accessory coupled to the portable radio, the remote accessory including:
a controller; and
an accelerometer coupled to the controller and monitoring the user's vital sign information;
a dispatch station for receiving the vital sign information from the radio; and
a detection notification device coupled to the dispatch station for detecting the vital sign information and providing notification of the vital sign information to others.

11. The communication system of claim 10, wherein accelerometer monitors at least one of heart rate, respiration.

12. The communication system of claim 10, wherein the remote accessory comprises a remote-speaker-microphone.

13. The communication system of claim 12, wherein the accelerometer monitors movement and at least one of heart rate and respiration.

14. The communication system of claim 12, wherein the accelerometer determines a movement waveform and wherein heart rate and respiration parameters are determined from the movement waveform.
